(19) United States
(12) Patent Application Publication
(10) Pub. No.: US 2016/0220500 A1
(43) Pub. Date: Aug. 4, 2016

(54) Targeting Intracellular Copper Ions for Inhibiting Angiogenesis Using Nanoparticles of Ternary Inorganic Metal Sulfide M1M2S4 (M1, independently, is Mg, Ca, Mn, Fe, or Zn; M2 = Mo or W) Compounds to Treat Metastatic Cancer

(71) Applicant: KENT STATE UNIVERSITY, Kent, OH (US)

(72) Inventors: Songping D. HUANG, Kent, OH (US); Gail C. FRAIZER, Hudson, OH (US); Vindya S. PERERA, Kent, OH (US)

(21) Appl. No.: 14/941,081

(22) Filed: Nov. 13, 2015

Related U.S. Application Data
(60) Provisional application No. 62/079,733, filed on Nov. 14, 2014.

(51) Int. Cl. A61K 9/51; A61K 33/32; A61K 33/26; C01G 39/06; C01G 45/00; C01G 49/12; C01G 41/00; A61K 33/24; C01B 17/42

(52) U.S. Cl. CPC A61K 9/51; A61K 33/24; A61K 33/32; A61K 33/26; C01B 17/42; C01G 45/006; C01G 49/12; C01G 41/006; C01G 39/06

(57) ABSTRACT

This invention describes a new type of covalent-network ternary inorganic metal sulfide compounds M₁M₂S₄ (M₁, independently, is, Mg, Ca, Mn, Fe, or Zn; M₂=Mo or W) and a process for preparing the biocompatible nanoparticles of such compounds. The nanoparticles are surface-modified with a capping agent and/or a biocompatible polymer and have the size from a few nanometers to several thousand nanometers. These nanoparticles are nontoxic and can be internalized by cells to deplete copper ions via a highly selective ion-exchange reaction between the intracellular copper ions and the divalent ion bound in the nanoparticles for the application of inhibiting angiogenesis in cancer and other diseases.

### FIELD OF INVENTION

The invention relates generally to novel covalent-network ternary inorganic metal sulfide compounds containing a divalent metal such as magnesium, calcium, manganese, iron or zinc and a hexavalent metal such as molybdenum or tungsten and sulfur that are useful in reducing intracellular copper concentrations for the application of inhibiting angiogenesis in cancer and other diseases.

### BACKGROUND OF INVENTION

Angiogenesis, also known as neovascularization, is the process of new blood Angiogenesis, also known as neovascularization, is the process of new blood vessel formation. In healthy adults, such process is tightly regulated and orchestrated by a variety of angiogenic factors and inhibitors in balance. Conversely, angiogenesis is a rate-limiting event in tumorigenesis, and thus the hallmark of cancer growth and metastasis. This concept has inspired researchers to search for angiogenic inhibitors for cancer treatment in the past three decades. Currently there are over 50 anti-angiogenic drugs for cancer treatment that are either on the market or at various stages of clinical trials in the US. All of these drugs, once considered very promising in cancer treatment, have failed to live up to the high expectations. The main problems with these angiogenic inhibitors are their limited efficacy, selective but nonspecific effects in different types of cancer, and they engender inherent or acquired resistance. There is an ongoing debate in the cancer research community on whether anti-angiogenic treatment of cancer using the current inhibitors triggers more invasive and metastatic tumors. This is because tumor angiogenesis is a complex and multistep process involving several parallel signaling pathways. There is increasing evidence to suggest that when one angiogenic signaling pathway is blocked, cancers can grow blood vessels using different angiogenic promoters to trigger other signaling pathways. Research to unravel the complexity of tumor angiogenesis and to develop new-generation drugs based on a better understanding of other signaling pathways will continue. In the meantime, there is an unmet medical challenge in current anti-angiogenic therapies that may benefit from more innovative approaches. The current invention is aimed at tackling the problem from a different angle by targeting the copper ion rather than the many cell-signaling biomolecules in tumor angiogenesis as a novel strategy for anti-angiogenic cancer treatment.

In 1980 McAuslan and Reilly found that copper salts are the simplest angiogenic components of tumor extracts that would stimulate the migration of endothelial cells in vitro. Over a decade later, copper was found to stimulate the proliferation and migration of endothelial cells in vitro. When a copper pellet was implanted in the stroma of the rabbit cornea model of angiogenesis, it would induce dose-dependent neovascularization. Recently, researchers have found that copper is a common co-factor to over two dozens of key angiogenic promoters or proteins involved in angiogenesis, including (1) angiogenin; (2) angiotropin; (3) ceruloplasmin; (4) collagenase; (5) FGF-1 (acidic); (6) FGF-2 (basic); (7) FGF receptor-1; (8) fibronectin; (9) gangliosides; (10) heparin; (11) Gly-His-Lys; (12) IL-1; (13) IL-6; (14) IL-8; (15) nuclear factor KB (NFκB); (16) prostaglandin (17) E-1; (18) synaptotagamin; (19) SPARC; (20) transforming growth factor β; (21) tumor necrosis factor α; and (22) VEGF. After these promoter molecules are activated, there is a specific requirement for copper in each step of migration, mitosis and differentiation of endothelial cells, and reshaping of matrix proteins into the tubular structure of micro-capillaries. Therefore, depletion of copper in cancer and vascular endothelial cells as well as in the cancer stromal microenvironment (SMT) will turn more than one angiogenic signal pathway quiescent, achieving a goal of using one stone to kill multiple birds.

Brem and co-workers explanted tumors into the brains of rats and rabbits and found that a mild D-penicillamine-induced copper deficiency greatly reduced the growth of the tumors and their invasiveness. However, when the D-penicillamine treatment was later extended to patients with brain tumor, no improvement in survival was found due partly to the fact that as a small molecule D-penicillamine is unable to cross the blood brain barrier (BBB) as well as its modest binding ability to copper ions. D-Penicillamine (D-PEN), i.e. (2S)-2-amino-3-methyl-3-sulfanyl-butanoic acid (Scheme 1) is an FDA-approved drug used to lower the excess copper accumulation in the liver of the patient with Wilson's disease (WD).

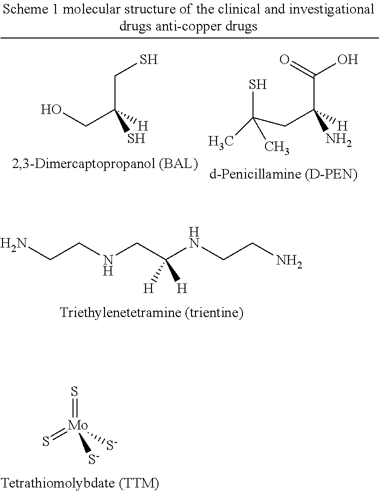

This disease is otherwise known as hepatolenticular degeneration, which is a recessive genetic disorder characterized by excess copper accumulation in the liver and other vital organs. Because WD is a debilitating disease, and if untreated, it can lead to severe disability, a need for liver transplantation, and death, there have been tremendous research efforts in developing clinical drugs in the form of chelation therapy for treating WD for the last seven decades. In 1951, the British anti-Lewisite (BAL) was introduced as the first clinical drug for WD. This chelating agent had been initially developed in World War II (WWII) as an antidote to the chemical warfare agent Lewisite and was later adopted for use in detoxifying heavy metal poisoning by arsenic, gold, antimony, lead or mercury (see Scheme 1). Because of some serious side effects including nephrotoxicity and hypertension of BAL, D-PEN, a metabolite of penicillin was introduced in 1956 as a better clinical drug for WD. In 1982, triethylenetetraamine (trientine; see Scheme 1), a less effective copper chelating agent than D-PEN, was introduced as a new clinical drug for WD, mainly for the patients who showed intolerance to D-PEN. Currently, the clinical use of triethylenetetramin is limited in the USA because such application has not been approved for the European market. In 1997, the US Food and Drug Administration (FDA) approved the use of zinc acetate as a clinical drug for WD. Unlike other three clinical drugs for WD, this compound is not a chelating agent, but zinc ions from the drug can stimulate the production of metallothionein in gut cells, which in turn binds copper ions to inhibit their absorption and transport to the liver. It has been shown that zinc acetate is only effective as a maintenance therapy for WD. Recently, tetrathiomolybdate (TTM; see Scheme 1) was introduced as an investigational drug for WD. Research has shown that TTM forms a non-bioabsorbable form of ternary complexes with copper and food proteins in the gastrointestinal tract to block the intestinal absorption of copper from the diet, thus creating a negative copper balance in the body. Among all these drugs for WD, D-PEN has the highest efficacy, and hence is currently the most widely used drug for WD across the world. However, the side effects of D-PEN are numerous, and several of these are severe. They include bone marrow and immune suppression, skin rash, mouth ulcers, nausea, and deterioration of various neurological functions. The latter side effect is believed to be caused by the ability of D-PEN to mobilize copper ions that are stored in the body tissues and reroute them into circulation, thus increasing the concentrations of copper in the brain. It has been estimated that about half of the WD patients treated with D-PEN would show neurologic deterioration, and a quarter of such patients would suffer irreversible neurologic damage for use of D-PEN. All of these side effects are attributable to the fact that this drug is delivered systemically with no organ-specificity, hence causing a variety of side effects due to the systemic toxicity of the drug. On the other hand, TTM is known to be susceptible to hydrolysis that releases hydrogen sulfide (H₂S) under the acidic conditions of the stomach via the reaction MoS₄²⁻+4H₂O→MoO₄²⁻+4H₂S. Although a small amount of H₂S is constantly produced in the human digestive tract from the anaerobic digestion of food and can be detoxified by several enzymes, this gas is considered more toxic than hydrogen cyanide (HCN) to the neural and circulating system. This fact suggests that TTM is unsuitable for the intravenous delivery as a drug. Furthermore, the manufacture, storage, and clinical use of TTM will be a safety concern. It should be noted that all these anti-copper drugs are based on small molecules or an ion (i.e. TTM). Although small molecules or ions are more likely to be water soluble for drug delivery via oral or intravenous administration, they all possess a common problem, that is the copper complexes formed from such soluble small molecules or ions are labile and can be re-partitioned between the biological fluids and various solid tissues to make copper the clearance of chelated copper from the body slow and incomplete. It is known that use of D-PEN for treating WD can often mobilize copper ions stored in the body tissues and reroute them into circulation to increase the concentrations of copper in the brain, causing a variety of neurodegenerative diseases. Furthermore, these small molecule-based drugs lack the ability or suitable mechanisms to penetrate cells to target intracellular copper ions for detoxification. They usually act to lower the systemic concentrations of copper ions in the body, making tissue- or organ-specific copper-depletion impossible. In general, depletion of the systemic rather than specific tissues or organs copper ions has a higher tendency to cause copper-deficiency related side effects in patients.

### SUMMARY OF THE INVENTION

The invention relates generally to novel covalent-network ternary inorganic metal sulfide compounds containing a divalent metal such as magnesium, calcium, manganese, iron or zinc and a hexavalent metal such as molybdenum or tungsten and sulfur that are useful in reducing intracellular copper concentrations for the application of inhibiting angiogenesis in cancer and other diseases.

Covalent network compounds or covalent network solids refer to chemical compounds in which the atoms are bonded by covalent bonds in a continuous network structure that extends throughout the entire substance. In a covalent network solid there are no individual molecules, and the entire structure may be considered a macromolecule. Moreover, they are not core-shell nanoparticles. The typical covalent network compounds are not soluble in water or any organic solvent, nor will they dissociate in solution to release soluble cations or anions.

Examples of covalent network compounds include diamond with a continuous network of carbon atoms and silicon dioxide or quartz with a continuous three-dimensional network of SiO₂ units. Examples of binary inorganic metal sulfide compounds with a covalent network structure include zinc sulfide found in nature as minerals sphalerite or wurtzite, iron disulfide found in nature as mineral pyrite, molybdenum disulfide found in nature as molybdenite and widely used as a solid lubricant in industry, and tungsten disulfide found in nature as tustenite and used in petroleum industry as a hydrodesulfurization catalyst.

The biggest advantage of using the ternary inorganic metal sulfide M₁M₂S₄ (M₁, independently, is, Ca, Mg, Mn, Fe, or Zn; M₂=Mo or W) as anti-copper drugs for targeting intracellular copper ions to treat metastatic cancer such as bladder cancer, breast cancer, colorectal cancer, kidney, lung cancer, melanoma, ovary cancer, pancreatic cancer, prostate cancer, stomach cancer, thyroid cancer and uterus cancer, is that through the ion-exchange reaction between the divalent metal ion (i.e. Mg²⁺, Ca²⁺, Mn²⁺, Fe²⁺ or Zn²⁺) and copper ions, another ternary metal sulfide compound Cu₂MoS₄ is formed and the biologically essential divalent metal ion Mg²⁺, Ca²⁺, Mn²⁺, Fe²⁺ or Zn²⁺ is released, achieving the net result of lowering the intracellular copper concentration while delivering a biologically essential divalent metal ion into the cell. The ternary metal sulfide Cu₂MoS₄ is also a covalent network compound in which copper ions are completely locked in their crystal lattice positions. This feature is in stark contrast from using soluble small molecules or ions as mentioned in the above to chelate copper ions, which leads to the formation of a copper complex that is usually labile and can be re-partitioned between the biological fluids and various solid tissues to make copper the clearance of chelated copper from the body slow and incomplete.

Another object of the present invention is to provide novel types of nanoparticles suitable for the intercellular depletion of copper for angiogenesis inhibition. Because these ternary metal sulfide compounds are insoluble in water, the nanoparticulate form of such compounds with proper surface coatings will impart water dispersability to the materials, and hence allowing for their in vivo delivery in patients via oral administration or intravenous injection.

Nanoparticles comprising a formula M₁MoS₄ or M₁WS₄ where M₁, independently, is, Mg, Ca, Mn, Fe,or Zn; and said nanoparticles, independently, have a diameter of from about 4 to about 900 nanometers.

A method for producing an angiogenic inhibitor for treatment of cancer and other diseases comprising appling nanoparticles of divalent metal M1, where M1, independently, is Mg, Ca, Mn, Fe or Zn, tetrathiomolybdate having the chemical formula M1MoS4, or a tetrathiotungstate having the chemical formula M₁WS₄, or both, to an animal.

A method for reducing intercellular copper concentrations in a human having cancer cells and/or human vascular endothelial cells, comprising the steps of forming a water dispersible covalent network of M₁MoS₄ or M₁WS₄ nanoparticles where M₁, independently, is, Mg, Ca, Mn, Fe or Zn; and administering an effective amount of said M₁MoS₄ or said M₁WS₄ nanoparticles to said human, said nanoparticles being capable of causing an ion exchange reaction whereby said copper is incorporated in said nanoparticles thereby depleting said copper ions from said cancer cells and/or said vascular endothelial cells, and inhibiting angiogenesis.

A method for reducing intercellular copper concentrations in a human having cancer cells and/or vascular endothelial cells, comprising the steps of forming a water dispersible covalent network of M₁MoS₄ or M₁WS₄ nanoparticles where M₁, independently, is, Mg, Ca, Mn, Fe or Zn, or any combination thereof; and administering an effective amount of said M₁MoS₄ or M₁WS₄ nanoparticles to effect cellular uptake of said M₁MoS₄ or M₁WS₄ nanoparticles into said cancer cells and/or said vascular endothelial cells and emitting copper therefrom.

A method for reducing cancer cells and/or vascular endothelial cells in a human, comprising the steps of forming a water dispersible extended covalent network of M₁MoS₄ or M₁WS₄ nanoparticles where M₁, independently, is Mg, Ca, Mn, Fe, or Zn, or any combination thereof; and treating said cancer cells and/or vascular endothelial cells with said M₁MoS₄ or M₁WS₄ compounds and reducing migration of said cancer or human vascular endothelial cells.

A method for making M₁M₂S₄ nanoparticles, comprising the steps of reacting a basic molybdenum sulfide or a basic tungsten sulfide in a solution of an amide with, independently, a magnesium salt, a calcium salt, a manganese salt, or an iron salt, in an aqueous solution containing a mercapto alkyl acid, and a basic hydroxide and producing said M₁M₂S₄ compound where M₁, independently, is Mg, Ca, Mn, or Fe, and M₂ is molybdenum, or tungsten.

### BRIEF DESCRIPTION OF DRAWING

FIG. 1 shows the TEM image of PVP-coated ZnMoS₄ NPs;

FIG. 2 shows the confocal-fluorescence (right) and bright-field (left) images of dye-labeled ZnMoS₄ NPs -treated HuVEC cells;

FIG. 3 shows the cell viability curve of PVP-coated ZnMoS₄ NP-treated HuVEC cells;

FIG. 4 shows inhibition of FGF2-induced tube formation by HuVEC cells using ZnMoS₄ NPs. Upper panels: bright-field and Calcein-stained images of HuVEC cells treated with FGF-2 in basal media, showing the tube formation. Lower panels: the corresponding images, showing nanoparticles' inhibition effect;

FIG. 5 shows the effect of ZnMoS₄ NP's inhibition on VEGF-induced tube formation in Huvecs. FIG. 5A. shows the absence of VEGF, i. e. only the basal medium is used; FIG. 5B. shows the presence of the basal medium treated with VEGF of 150 uL of 50 ng/ml solution; FIG. 5C. shows a treatment with a high dose of ZnMoS₄ of 150 uL of 50 ng/ml solution; FIG. 5D. shows a treatment with a low dose of ZnMoS₄ of 150 uL of 10 ng/ml solution; and FIG. 5E. shows a treated with 5 uM Sulforaphane;

FIG. 6A shows Huvec migration inhibited by ZnMoS₄ NPs in the presence of VEGF induced Huvec cells; and FIG. 6B shows Huvec migration inhibited by ZnMoS₄ NPs in the presence of FGF-2 induced Huvec cells;

FIG. 7A shows ZnMoS₄ NPs decreases Huvec migration potential at basal Oh and basal 16 h; FIG. 7B shows ZnMoS₄ NPs decreases Huvec migration potential at VEGF 0 hand VEGF 16 h; FIG. 7C shows ZnMoS₄ NPs decreases Huvec migration potential at ZnNPs low dose 0 hand ZnNPs low dose 16 h; FIG. 7D shows ZnMoS₄ NPs decreases Huvec migration potential at ZnNPS high dose 0 hand ZnNPs high dose 16 h; and FIG. 7E shows overall 0 hvs. 16 h Huvec migration;

FIG. 8A shows viability of Huvec cells; FIG. 8B shows viability of PC3 cells; and FIG. 8C shows the viability of LNCaP cells;

FIG. 9 shows ZnMoS₄ NPs down-regulated VEGF expression in PC3 prostate cancer cells at both mRNA and protein level; and the results from the Western blot with using a polyclonal VEGF antibody and anti-rabbit HRP-conjugated secondary antibody; and.

FIG. 10A shows PC3 tumor weights not strongly affected by Zn-NPS, but deviation increased; FIG. 10B shows PCT3 tumor volume not strongly affected by Zn-NPS, but deviation increased; FIG. 10C shows VEGF expression in mice tumor.

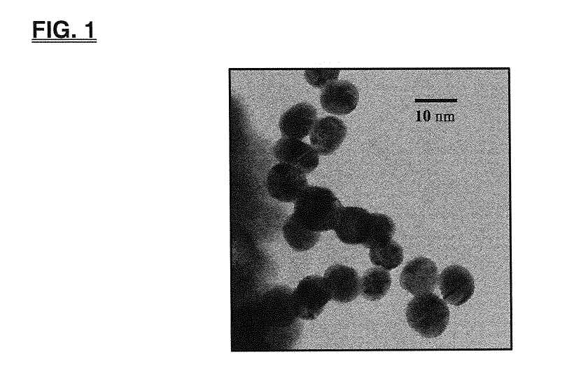

### DETAILED DESCRIPTION OF INVENTION

The preparation of the nanoparticles of the present invention can be carried out in the following manner.

The main object of the present invention is to provide a novel type of nanoparticles suitable for intracellular depletion of copper for angiogenesis inhibition. The typical preparation can be carried out as follows: from about 0.1 mL to about 300 mL, and desirably from about 1 mL to about 100 mL, and preferably from about 25 mL of various mercapto alkyl acids having a total of from 1 to about 50 carbon atoms, desirably from about 1 to about 12 carbon atoms, and preferably 3-mercaptopropionic acid was added to about 1 mL to about 200 mL, desirably from about 2 mL to about 100 mL, and preferably 10 mL of about 0.01 N to about 18 N, desirably from about 0.1 N to about 10 N, and preferably 1N NH₄OH solution. Other suitable hydroxides include NaOH, KOH, Ca(OH)₂, or Na₂CO₃. Then an effective amount of an M₁ salt is added to water. Suitable M₁ salts include zinc acetate, zinc chloride, zinc sulfate, zinc perchlorate, zinc nitrate; as well as non-zinc salts such as magnesium acetate, magnesium chloride, magnesium sulfate, magnesium perchlorate, magnesium nitrate; calcium acetate, calcium chloride, calcium sulfate, calcium perchlorate, calcium nitrate; manganese acetate, manganese chloride, manganese sulfate, manganese perchlorate, manganese nitrate; iron(II) acetate, iron(II) chloride, iron(II) sulfate, iron(II) perchlorate, iron(II) nitrate, or any combination thereof. Suitable amounts of M₁ salts range from about 1 to about 250 mg, desirably from about 100 to about 200 mg, and preferably about 110 to about 150 mg. Thus, about 130 mg of Zn(O₂CCH₃)₂(H₂O)₂ was added to about 0.1 mL to about 300 mL, desirably from about 1 mL to about 100 mL, and preferably 6 mL of water was added dropwise to the above mixture. Then a solution of from about 1.0 mg to about 400 mg, desirably from about 10 mg to about 300 mg, and preferably 130 mg of a basic molybdenum sulfide (NH₄)₂MoS₄, or (NH₄)₂WS₄ was added to about 0.1 mL to about 500 mL, desirably from about 1 mL to about 100 mL, and preferably about 28 mL of a mixture of formamide and water (volume ratio from about 0.1 to about 100, desirably from about 1 to about 20, and preferably from 1:14). This solution of the noted sulfide compounds with water and formamide was added dropwise to the above mixture with vigorous stirring resulting in a color change to yellowish brown. Finally, from about 1 to about 10, desirably from about 2 to about 6, and preferably 3.00 g of PVP (polyvinylpyrrolidone) or other equivalent dispersable polymer having a molecular weight of about 8,000 was added to the reaction mixture and the stirring was continued at room temperature for about 0.1 to about 72, desirably from about 1 to about 24 and preferably 6 hrs. The reaction mixture was then dialyzed using a cellular-membrane bag from about 800 to about 20,000, desirably from about 1,200 to about 12,000, and preferably a molecular weight of about 3,000 in distilled water and lyophilized to give light brown powder.

In a similar manner various other angiogenesis inhibiting inorganic sulfide-based compounds can be formulated utilizing either molybdenum or tungsten along with the various noted salts such as magnesium, calcium, manganese, or iron, in lieu of zinc. That is, similar ratios of the various compounds to one another utilizing essentially the same noted process steps will result in M₁M₂S₄ compounds that are novel and never heretofore produced wherein M₁ is magnesium, calcium, manganese, or iron, and M₂ is molybdenum or tunsten. Thus, in any remaining portion of the present specification whenever zinc is utilized, compounds containing either calcium, manganese, magnesium, or iron can be substituted and utilized therefore. The size of the M₁M₂S₄ nanoparticles of the present invention generally range from about 4 to about 900 nanometers, desirably from about 10 to about 300 nanometers, and preferably from about 15 to about 200 nanometers.

The M₁M₂S₄ nanoparticles of the present invention are desirably capped or contain a coating agent, i.e, a capping agent such as a biocompatible polymer, or a water soluble polymer, or any combination thereof. Examples of biocompatible polymers include dextran, polyethylene glycols, and other polymers of glucose. Examples of water soluble polymers include polyvinyl acetate, polyvinyl alcohol, and the like. A preferred polymer is polyol(N-vinylpyrrolidone).

To evaluate the selectivity of different metal ternary sulfide M₁M₂S₄ compounds including MgMoS₄, MgWS₄, CaMoS₄, CaWS₄, MnMoS₄, MnWS₄, FeMoS₄, and FeWS₄ with respect to Cu²⁺ ions in aqueous solution, a 100-mg sample of the noted sulfide compounds were first ground into fine powder, and sealed in a dialysis bag which was then soaked in a solution containing Cu²⁺ ions at a 50 ppm level. After 24 hours of incubation time, an aliquot of solution was taken out, diluted with 2% HNO₃ acid and analyzed by atomic adsorption spectrometry to determine the concentration of the copper metal ion. The difference in concentrations of the copper metal ions before and after the removal is expressed as percent removal of the copper metal ion. The results are given in Table 1.

TABLE 1
Percent removal of the copper ion by various M₁MoS₄ and
M₁WS₄ (M₁ = Mg, Ca, Mn, Fe; M₂ = Mo or W).

| Compound | % Cu removal |
|---|---|
| MgMoS₄ | 33 |
| MgWS₄ | 30 |
| CaMoS₄ | 31 |
| CaWS₄ | 30 |
| MnMoS₄ | 36 |
| MnWS₄ | 32 |
| FeMoS₄ | 47 |
| FeWS₄ | 49 |

The above percent removal of copper ion generally ranges from about 30 to about 50% copper ion removal. These values are considered to be very good inasmuch as significant amounts of copper ion were removed that correlate to removal from a human body. That is, they remove harmful, excessive amounts of copper. Higher removal amounts are not desired since copper is necessary for survival and high removal amounts could injure a person, or perhaps even result in death.

The M₁MoS₄ or M₁WS₄ copper depleting compounds of the present invention can be added to a human being by generally any conventional manner. Thus, such substances can be added orally as by way of being contained in water. Alternatively, they can be injected intravenously as into a blood vessel, or alternatively as into a muscle. Once intercellular copper concentrations in a human being have been extracted from cancer cells, and/or vascular endothelial cells, they are excreted at a natural manner, such as by urination or defecation.

Bioassay Results:

The reaction mixture was dialyzed using a cellular-membrane bag (MWCO=3,000) in distilled water and lyophilized to give light brown powder. Transmission electronic microscopy (TEM) images of the PVP-coated nanoparticles revealed spherical nanoparticles with a narrow distribution of size at ca. 8±2 nm (FIG.

1).

The cellular uptake of PVP-coated ZnMoS₄ in human vascular endothelial cells (HuVEC) was confirmed using the confocal fluorescence microscopic technique. Nanoparticles were first conjugated the fluorescence dye carboxyfluorescein before incubating with cells. The fluorescent images of the live HuVEC cells treated with the dye-labeled nanoparticles showed strong fluorescent signals in the perinuclear region of the cell, indicating an untargeted distribution of nanoparticles in the cytoplasm without specific binding to any of the small organelles in the region. This observation suggests that the cellular uptake of these nanoparticles is via endocytosis (FIG. 2).

### EXAMPLE 1

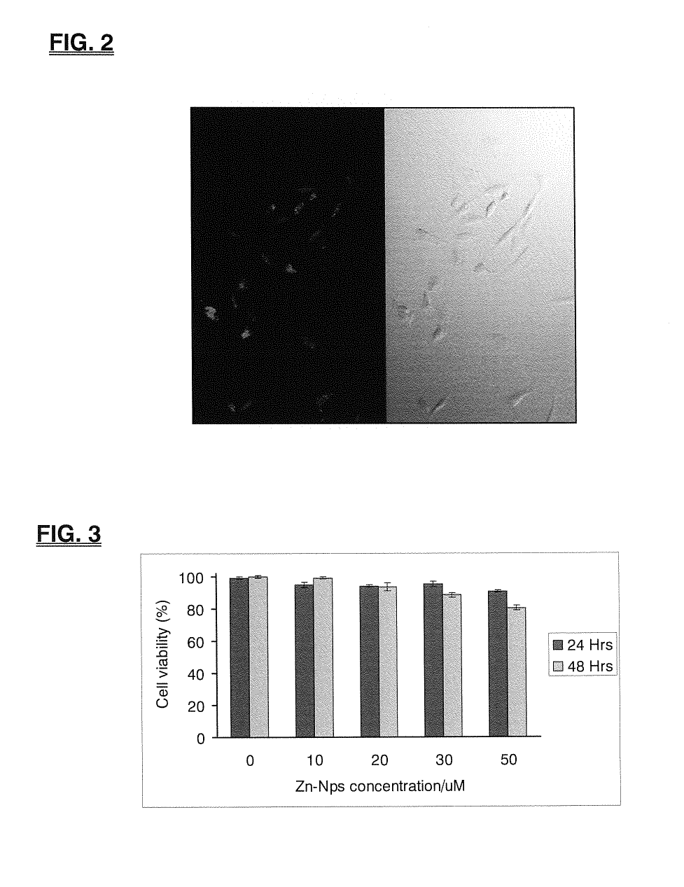

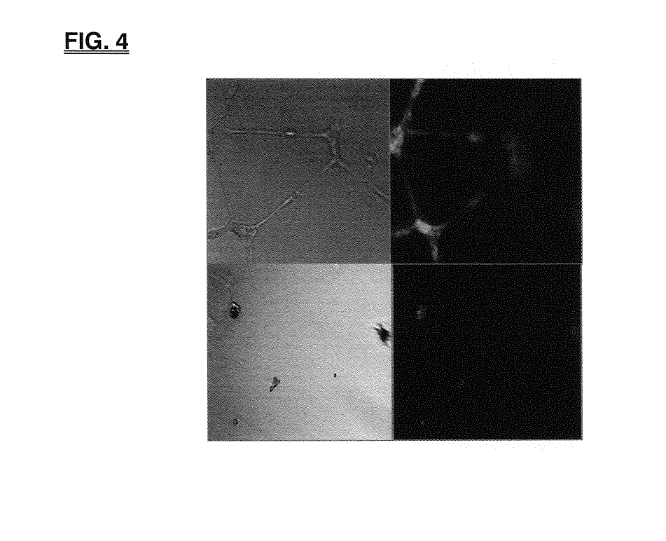

The cell viability assay was carried out using the MTT method. HuVEC cells were seeded in a 96-well plate at a density of 1×10⁴ cells per well with endothelial cell basal growth medium-2 (EBM-2) medium containing 10% FBS (fatal bovine serum) plus 1% penicillin-streptomycin and incubated for 5 hours at 37° C. in an atmosphere of 5% CO₂ and 95% air to allow cells to attach to the surface. Cells in each well were then incubated with 100 μL of fresh medium containing various concentrations of the nanoparticles for 24 hours and 48 hours. Control wells contained the same medium without nanoparticles. Each concentration was tested in replicates of three. At the end of the incubation period, 10 μL of 5 mg/mL 3-(4,5-dimethylthiazol-2-yl)-2,5-diphenyltetrazolium bromide (MTT) was added to each well and incubated for another 3 hrs. Then 100 μl of detergent reagent was added to each well and incubation was continued for another 4 hrs at 37° C. Finally the absorbance was determined at 570 nm using BIORAD ELISA plate reader. The assay results were presented as percent viable cells. Viability percentage was determined from the ratio of the absorbance of the treated cells to the untreated controls. The results indicate that after 48-hr incubation with 50 uM nanoparticles the cell viability remained high (>79%) (FIG. 3).

The inhibition of angiogenesis by ZnMoS₄ nanoparticles was demonstrated using an in vitro model system for angiogenesis. Specifically, induction of tube formation by HuVEC cultured on basement membrane extracts was employed as the model. Following induction by fibroblast growth factor 2 (FGF2) and vascular endothelial growth factor (VEGF) treatment, outgrowth and branching of HuVEC cells was measured in the presence or absence of ZnMoS₄ nanoparticles. The results clearly showed that the copper depleting ZnMoS₄ nanoparticles suppressed FGF2 induction of tube formation and branching by HuVEC cells (FIG. 4).

### EXAMPLE 2

Nanoparticles of ZnMoS₄ and M₁M₂S₄ (M₁, independently, is Mg, Ca, Mn, or Fe; M₂=Mo or W) compounds inhibit endothelial cell tube formation in the in vitro model of angiogenesis. All these compounds can act to lower the copper concentration in the endothelial cells used in this model study as well as the copper concentration in the culture media by the ion-exchange with the divalent ion in the ternary compounds via the following reaction:

Cuⁿ⁺ (n=1 or 2)+M₁M₂S₄→CuM₂S₄+M₁²⁺.

Since copper is a required co-factor for many angiogenesis growth factors including VEGF, bFGF, angiogenin in the formation of endothelial cell tubes, the angiogenesis is therefore inhibited.

Further testing was conducted as follows:

The tube formation assay is an in vitro a model of angiogenesis commonly used to measure the ability of endothelial cells to form “tubes” (i.e. three-dimensional structures that resemble vessel walls). Tube formation studies were conducted in a 96-well plate format using an in vitro angiogenesis assay kit from Trevigen Inc. (Trevigen, Gaithersburg, Md.). Prior to tube formation assay, Huvec cells were starved overnight in EGM-2 basal medium in the culture dish. To prepare the chambers for the assay, basement membrane extract (BME) solution was thawed in ice-water bath at 2-4° C. in a refrigerator overnight, and then 50 μl of BME solution was aliquoted into each well of a 96-well plate and incubated for 1 hour at 37° C. to gel. The cells were harvested and counted, and a single cell suspension at 1×10⁶ cells/ml was prepared. The cells were diluted in EBM basal medium in the presence or absence of angiogenesis inducers VEGF (50 ng/ml) or FGF2 (50 ng/ml) and nanoparticle inhibitors of M₁M₂S₄ or sulfophorane (5 uM). The cells were added at a density of 1×10⁴ per 100 ul to each well, without disturbing gelled BME and incubated for 16 h in a CO₂ incubator at 37° C. HuVEC cells were incubated for 30 min with Calcein AM (2 uM) at 37° C. for staining of live cells for imaging. Tube formation was visualized using a fluorescence microscope (485 nm excitation/520 nm emission) at 200× total magnification. Numbers of branch points were counted for each of six randomly chosen fields, and then averaged for each condition. The experiment was reproduced twice. Statistical significance was determined using student t-test.

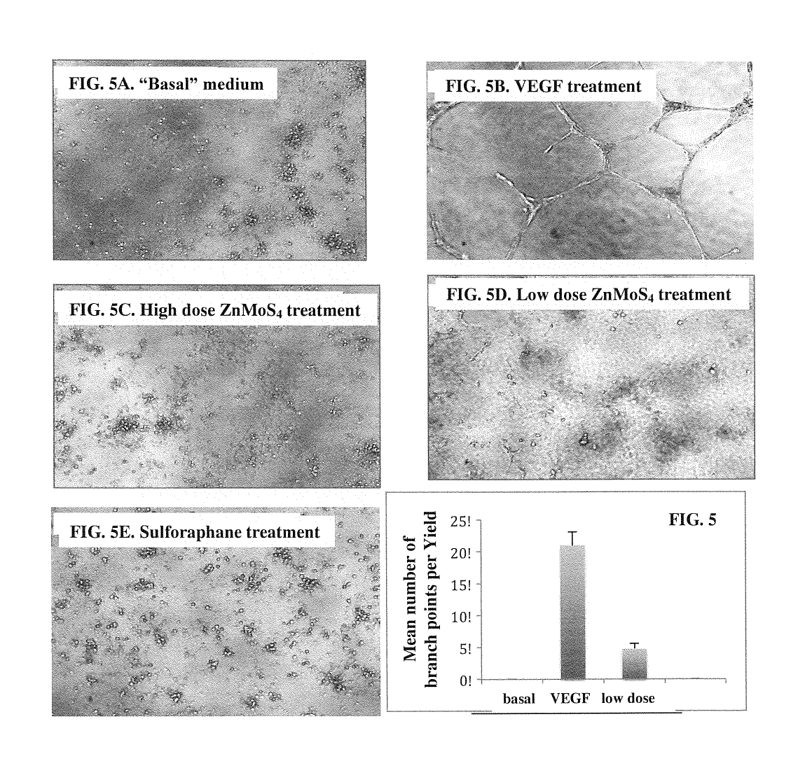

Since the tube formation assay is a measurement of the ability of endothelial cells to form three-dimensional structures that resemble blood vessels under VEGF treatment, using Huvec cells, it is demonstrated that all the divalent metals including magnesium, calcium, manganese, iron and zinc in combination with either molybdenum or tungsten in the ternary metal sulfide M₁M₂S₄ compounds are effective in inhibiting endothelial cell tube formation in the above mentioned bioassays. For example, more qualitative measurements of the inhibitory effect using nanoparticles of ZnMoS₄ NPs showed the following results: VEGF (50 ng/ml) caused marked increase in endothelial cell tube formation and branching points compared with basal medium alone (FIGS. 5A and 5B). However, in contrast, when Huvec cells were treated with VEGF and a high dose (50 ng/ml) of nanoparticles or a low dose (10 ng/ml) of nanoparticles of ZnMoS₄, there was inhibition of tube formation (FIGS. 5C and 5D) in both doses and reduction of branching points (FIG. 5F). Similarly, sulforaphane (a known angiogenesis inhibitor) also prevented tube formation and was used as a negative control in our studies (FIG. 5E). The tube formation was quantified and expressed as mean number of branching points per viewing field as shown in FIG. 5F for the high dose treatment using nanoparticles of ZnMoS₄.

As shown in FIG. 5, confluent Huvec were starved for growth factors in EBM-2 basal medium overnight. The Huvec cells were harvested, counted, and diluted in EBM-2 basal medium in the presence (Panels B-E) or absence (Panels A) of VEGF (50 ng/ml) and ZnMoS₄ NPs at 50 ng/ml (Panel C) and 10 ng/ml (Panel D). The cells were seeded on gelled BME in 96-well plate and incubated for 24 h in a 5% CO₂ incubator at 37° C. Panel E shows VEGF induced cells treated with angiogenesis inhibitor sulforaphane (5 uM). The tube formation was visualized under bright field microscope, and photomicrographs were acquired. Representative photomicrographs are shown (magnification 100×). F. Quantitative assessment of the tube formation was done by counting the average number of branch points per viewing field. The bars represent mean±SD (n=6); statistical significance was determined by student t-test p<0.05 VS VEGF.

### EXAMPLE 3

nanoparticles of ZnMoS₄ and M₁M₂S₄ (M₁, independently, is, Mg, Ca, Mn, or Fe; M₂=Mo or W) compounds decrease the migration of Huvec endothelial cells. Endothelial cell migration is essential to angiogenesis. Endothelial cell migration is directionally regulated by chemotactic, hapotactic, and mechanotactic stimuli and further involves degradation of extracellular matrix to enable progression of the migrating cells. An in vitro migration bioassay called the Boyden chamber assay was used to study the effect of nanoparticles on the migration of Huvec endothelial cells. Specifically, Huvec cells were grown in EGM-2 growth medium in 35 mm tissue culture dish until 80-90% confluent. The cells were starved 24 hours in EBM-2 basal medium prior to harvesting, counting and resuspending at 1×10⁶ cells/ml in EBM-2 basal medium. 50 ul of cell suspensions were added to the top chamber, along with any listed angiogenesis inhibitors, a low dose of nanoparticles (10 ng/ml), a high dose of nanoparticles (50 ng/ml), or the control using sulforaphane (5 uM) were introduced to the cell cultures. In the bottom chamber of each well was added 150 ul of medium containing chemoattractants, either VEGF (50 ug/ul), or FGF-2 (50 ug/ul); and the same inhibitors described above. Cells were allowed to migrate for 24 hours in a 37° C. CO₂ incubator.

The top chambers and bottom chamber were washed with 100 ul 1× washing buffer (Trevigen Migration Assay Kit), then 100 ul of crystal violet was added to the bottom chamber to stain migratory cells and cells were incubated at 37° C. CO₂ incubator for 30 minutes. 100 ul of cell dissociation solution was added to the bottom chamber of assay plate, and incubated for 30 minutes. The absorbance of the stained cells in the bottom chamber was read at OD 560 nm. The relative absorbance was converted to cell numbers using a standard curve previously determined for our Huvec cells by measuring the absorbance at OD 560 nm for known numbers of Huvec cells. The percentage of cells that migrated was determined by calculating the number of migrating cells divided by the number of total cells loaded into the upper chamber. The bars represent mean±SD (n=3); statistical significance was determined by student t-test p<0.05.

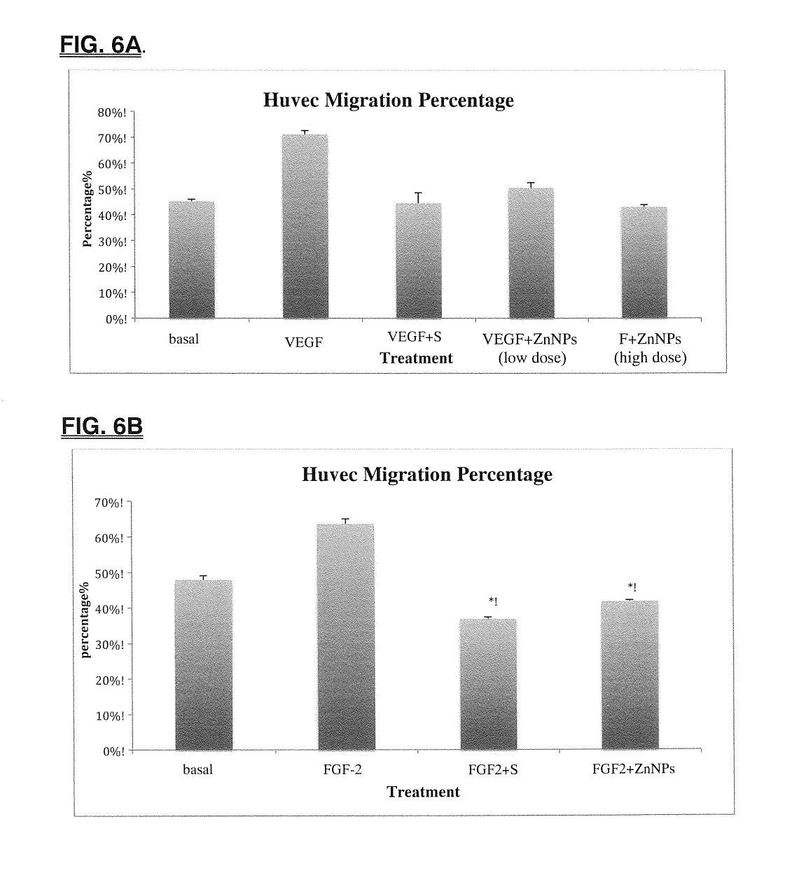

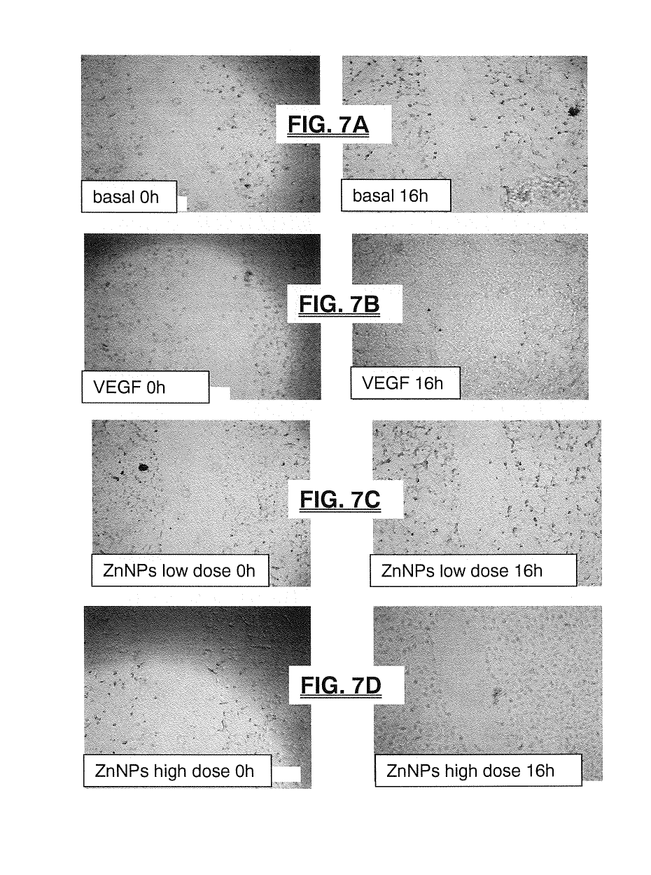

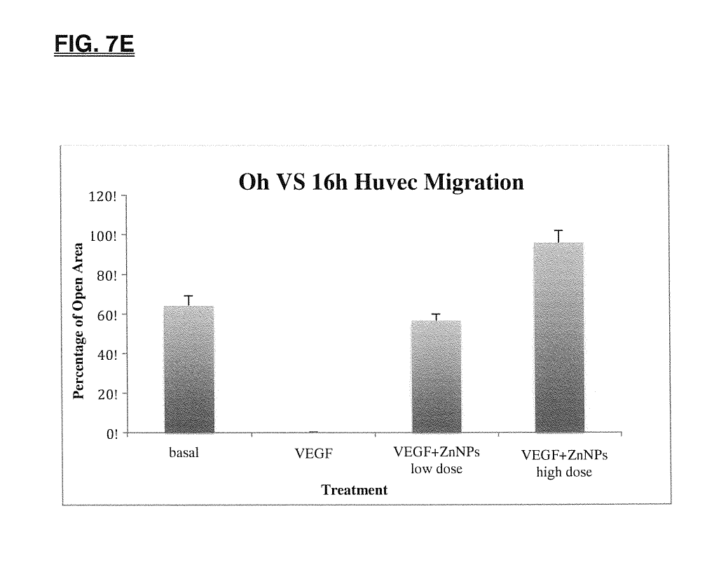

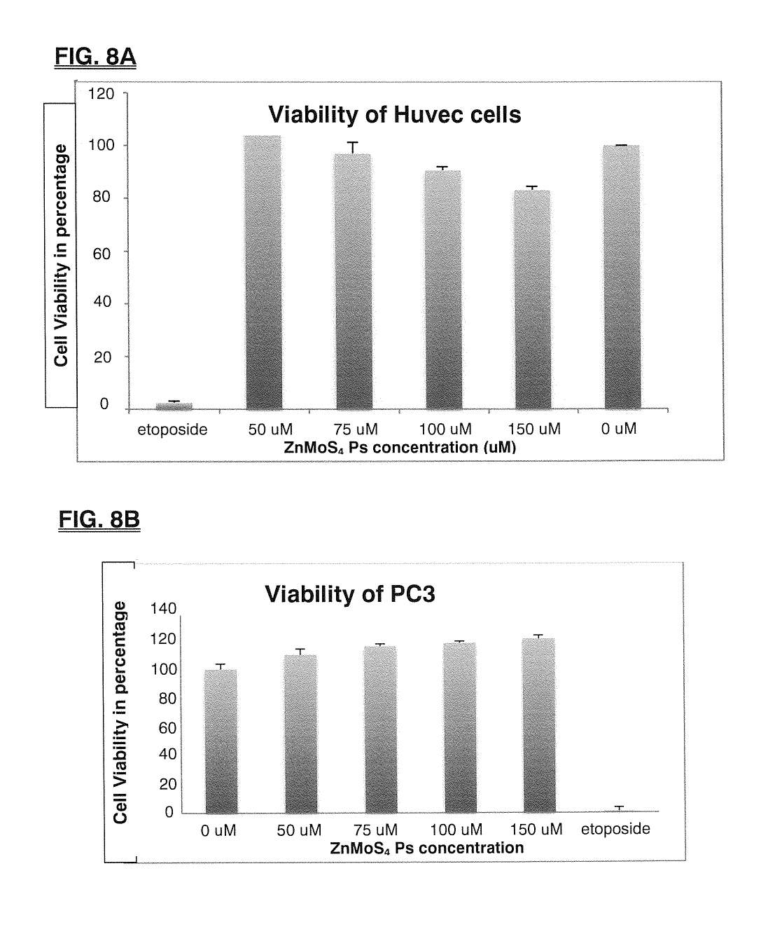

The reduction in migration of Huvec endothelial cells was observed when the divalent metals including magnesium, calcium, manganese, iron and zinc in combination with either molybdenum or tungsten in the ternary metal sulfide M₁M₂S₄ compounds were used in this bioassay. Here, focus was on quantitatively measured results obtained from the use of nanoparticles of ZnMoS₄. First, was assessed the VEGF (50 ug/ml) induced cell migration characteristics of Huvec cells seeded in the presence or absence treatment with a high dose (50 ng/ml) or a low dose (10 ng/ml). Migrating cells were stained with crystal violet and absorbance of dissociated cells was measured at OD560, percentage of migrating cells was determined for triplicate wells and average is shown for each treatment. As shown in FIG. 6A, both high dose and low dose of ZnMoS₄ treatment decrease the number of Huvec cells migrating compared non-treated cells. This inhibitor was similar to that produced by 5 uM sulforaphane. The results were confirmed using FGF2 (50 ug/ml) as another angiogenesis inducer (FIG. 6B). An alternative method was also used to test the effect of ZnMoS₄ on Huvec migration using the so-called “wound healing” assay where cells fill in a scratch in a monolayer of cells. A wound was scraped with a sterile 1000 ul pipette tip across the middle of confluent monolayer of Huvec cells. Cells were incubated in basal medium alone (FIG. 7A) or with VEGF (50 ug/ml) (FIG. 7B) for 16 hours with or without ZnMoS₄ NPs treatment. Photos were taken at the time of scratch (left panels) and 16 hours (right panels) and images analyzed to determine the percentage of wound remaining open at 16 hours, using Image J. The results showed that VEGF treated Huvec cells migrated and filled in most of the wounded area. In contrast, low dose ZnMoS₄ NPs decreased the migration of Huvec cells compared to VEGF only (FIG. 7C) and migration was completely blocked at high dose (FIG. 7D). These results demonstrated that ZnMoS₄ NPs were able to decrease the migration potential of Huvec cells triggered by VEGF.

FIG. 6—ZnMoS₄ NPs treatment inhibited VEGF and FGF-2 induced Huvec cells migration. Huvec cells (5×104 cells/well) in basal media were seeded in to the top chamber along with angiogenesis inhibitors sulforaphane (5 uM) or with ZnMoS4 NPs treatment at either low dose (10 ng/ml) or high dose (50 ng/ml); medium was added to the bottom chamber with or without FGF-2 (FIG. 6B) or VEGF (FIG. 6A), and included ZnMoS₄ NPs at either high dose (50 ng/ml) or low dose (10 ng/ml). Average percentage of migrating cells was calculated as described in the text. The experiment was done in triplicate. Significance was determined by student's t-test (p<0.05).

FIG. 7—ZnMoS₄ NPs decreases Huvec migration potential. The confluent Huvec cells were wounded with a pipette tip (Panel A-D) and incubated with basal media alone (Panel A) or with 50 ng/ml VEGF (Panel B-D) and treated with ZnMoS₄ NPs either at low dose (10 ng/ml) (Panel C) or high dose (50 ng/ml)(Panel D) for 16 h. The “wounded” areas were photographed at 0 h and at 16 h. A representative photomicrograph is shown for each condition (Magnification ×100). Area of “wound” remaining open was measured by Image J, and the average open area of four images was determined. Panel E shows quantitative assessment of the cell migration as percent mean open area of migration. The bars represent mean±SD (n=4); **p<0.05 Vs VEGF.

### EXAMPLE 4

nanoparticles of ZnMoS₄ and M₁M₂S₄ (M₁, independently, is, Mg, Ca, Mn, or Fe; M₂=Mo or W) compounds are nontoxic to vascular endothelial cells and prostate cancer cells (i.e. PC3 and LNCaP cells). The MTT cell proliferation assay (Trevigen®) was used to measure cell viability and cell proliferation in order to quantify the cell population's response to nanoparticles of ZnMoS₄ and M₁M₂S₄ (M₁, independently, is, Ca, Mn, or Fe; M₂=Mo or W) compounds. It was tested whether these NPs had negative effects on proliferation and viability of the above three cell lines. The MTT cell viability assay is based on the absorbance of dissolved MTT (tetrazolium salt 3-[4,5-dimethylthiazol-2yl]-2,5-diphenyl-tetrazolium bromide) formazan crystals formed in living metabolically active cells, which is proportional to the number of viable cells. Single cell suspensions at 106 per mL were seeded (10⁴ cells per well) in 96-well plate. The NPs were added at different concentrations (0-150 uM) in basal medium to bring the total volume to 100 ul per well. As a control a cytotoxic dose of etoposide was added to some wells in the place of the NPs. Cells were incubated overnight and then MTT reagent and detergent was added to each well and cells were incubated for 2 more hours in the dark. The absorbance in each well was measured at 570 nm in a microplate reader and the average values from triplicate wells were determined by subtracting the average values for blank of each treatment condition. The percentage of live cells was determined by calculating the absorbance of NPs treated wells divided by the absorbance of untreated wells. Student t-test was performed to determine significance.

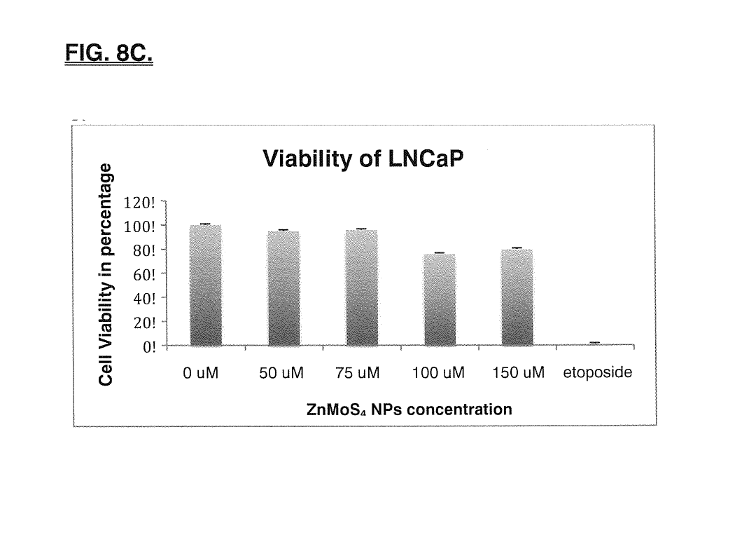

It was found that nanoparticles of M₁M₂S₄ (M₁, independently, is, Mg, Ca, Mn, Fe, or Zn; M₂=Mo or W) compounds are nontoxic to the three cell lines assayed. The representative data given in the following are obtained from the use of nanoparticles of ZnMoS₄. First, nanoparticles of ZnMoS₄ NPs did not reduce Huvec cell viability over a broad range of concentration. Particularly noticeable is that larger than 80% cell viability was found when the cells were treated with 150 uM ZnMoS₄ NPs (FIG. 8A). Second, nanoparticles of ZnMoS₄ NPs did not reduce the cell viability of PC3 and LnCaP cells. Similarly, larger than 80% cells were found to be viable at 150 uM ZnMoS₄ NPs for both PC3 and LNCaP cells (FIGS. 8B and 8C). In conclusion, ZnMoS₄ NPs were non-toxic to Huvec and prostate cancer cell lines, suggesting that ZnMoS₄ NPs inhibit tube formation without killing the cells.

FIG. 8: ZnMoS₄ NPs are not cytotoxic for Huvec endothelial cells and prostate cancer cells. A. Viability of Huvec cells after incubation with ZnMoS₄ NPs or etoposide for 24 hrs. was measured by MTT assay. Shown is percent viability compared to cells treated with diluent alone (0 uM ZnMoS₄ NPs). Viability of PC3 (Panel B) and LNCaP (Panel C) were also measured after incubation with ZnMoS₄ NPs or etoposide for 24 hrs. described as A. Treatment with a cytotoxic dose of etoposide was used as a cytotoxic positive control. The bars represent mean±SD (n=3); statistical significance was determined by student t-test, there was no statistical difference between the viability of ZnMoS4 NPs treated cells and untreated controls.

### EXAMPLE 5

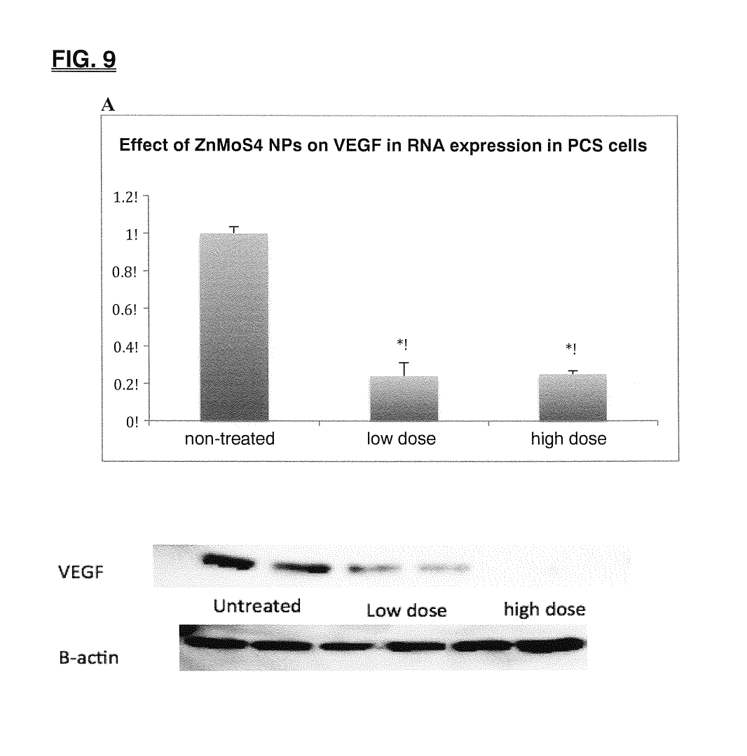

nanoparticles of ZnMoS₄ and M₁M2S₄ (M₁, independently, is, Mg, Ca, Mn, or Fe; M₂=Mo or W) compounds down-regulated VEGF expression both at the mRNA and protein level. To determine whether VEGF mRNA expression was decreased by ZnMoS₄ NPs treatment, confluent monolayer of PC3 cells were serum starved overnight and then treated with either high dose (50 ng/ml) or low dose (10 ng/ml) ZnMoS₄ NPs for 24 hours. FIG. 9A shows response of VEGF mRNA expression to ZnMoS₄ NPs measured by Taqman quantitative PCR and normalized to 18S mRNA. As shown in FIG. 9A, VEGF expression was significantly decreased at both low dose and high dose of ZnMoS₄ NPs treatment compared with untreated control. Given that mRNA was decreased after ZnMoS₄ NPs treatment, VEGF protein expression was examined in PC3 cells as well by following the ZnMoS₄ NPs treatment at both low dose and high dose. As shown in FIG. 9B, VEGF protein expression was decreased after ZnMoS₄ NPs treatment at low dose and was undetectable after high dose treatment for 24 hours.

FIG. 9: ZnMoS₄ NPs down-regulated VEGF expression in PC3 prostate cancer cells at both mRNA and protein level. (A) VEGF mRNA expression in PC3 cells treated with ZnMoS₄ NPs at low dose (10 ng/ml) and high dose (50 ng/ml) measured with Taqman q RT PCR. 18S rRNA expression was used to normalize VEGF expression.Values represent fold change relative to untreated controls. A student t-test was performed and significance was determined. (*p<0.05). (B) VEGF protein expression in PC3 cells treated 24 hours with ZnMoS₄ NPs at low dose (10 ng/ml) and high dose (50 ng/ml). Western blot with 40 ug of protein were probed with polyclonal VEGF antibody and anti-rabbit HRP-conjugated secondary antibody. Proteins were visualized by ECL incubation and Fuji LAS 3000 detection system. Blots were stripped and reprobed with b-actin antibody as loading controls.

### EXAMPLE 6

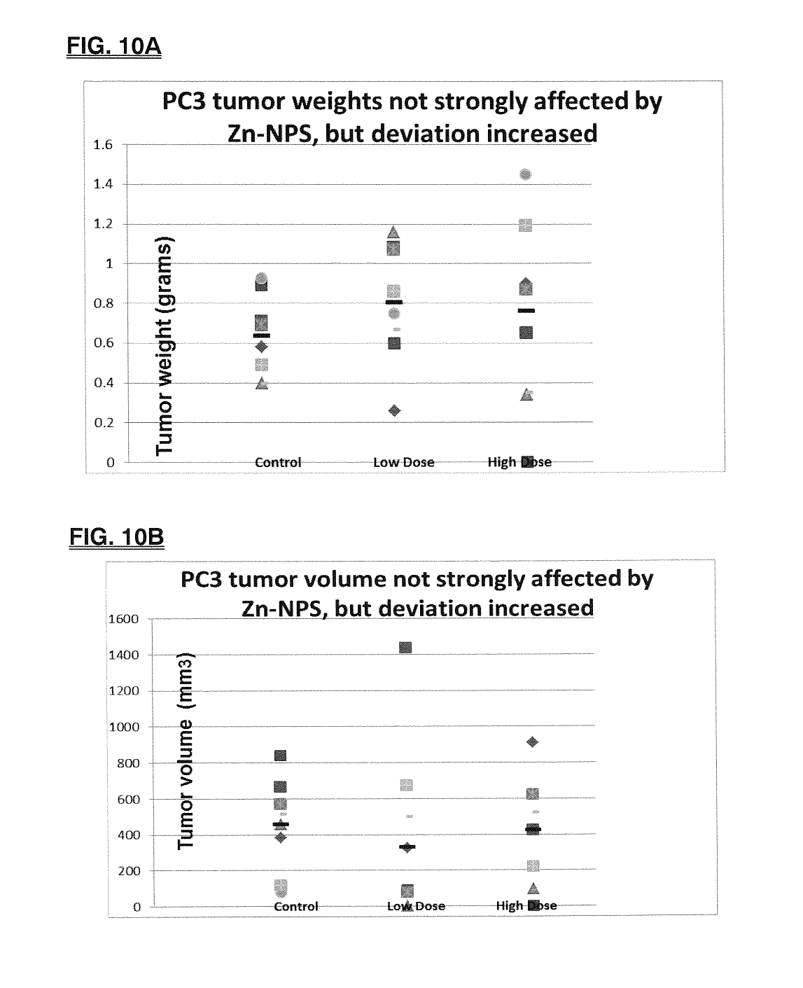

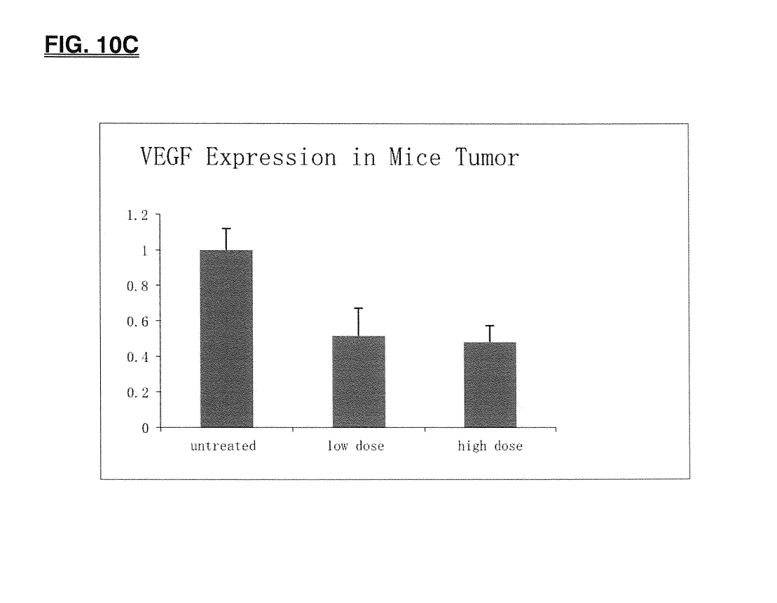

ZnMoS₄ NPs reduce VEGF expression without affecting tumor growth of PC3 xenografts. The tumor therapy of ZnMoS₄ NPs was examined in immunocompromised male mice (nu/nu strain, Jackson Laboratory) by monitoring tumor growth and angiogenesis in such animals. Approximately 6×10⁶ PC3 cells were suspended in 0.1 mL of sterile serum free culture medium and then injected subcutaneously into the right flank of 24 male nude mice. Forth-eight hours after tumor injection, mice were treated in 3 groups by I.P injection of Group 1 sterile 0.1 ml PBS control; Group 2 a mixture of ZnMoS₄ NPs in PBS at high dose (2 mg/mouse); Group 3 ZnMoS₄ NPs at low dose (0.2 mg/mouse). Tumor sizes were measured with microcalipers every week and tumor volumes calculated by the formula: length×width²×0.5236. After 28 days, or if tumor volume>500 mm³, mice were euthanized and tumors were collected and weighed. Results showed that ZnMoS₄ NPs did not decrease mean or median tumor weight (FIG. 10A) or tumor volume (FIG. 10B). However, a large variation was observed in tumor weights in the two treatment groups (low dose 10 mg/kg and high dose 100 mg/kg group). For mice treated at high dose of ZnMoS₄ NPs, smaller weight tumors were observed more frequently than in control groups. Thus, the deviation of tumor weights may have been associated with treatment. However, tumor volumes measured with microcalipers did not show this variation. These observations are consistent with the fact that ZnMoS₄ NPs are not toxic to PC3 cells, thus cannot function as an anti-cancer drug. However, levels of angiogenesis indicated by VEGF expression were affected by treatment, that is ZnMoS₄ reduced VEGF mRNA expression in both low and high dose treatment groups as quantified by real-time PCR (FIG. 10C), confirming that this drug can inhibit angiogenesis in the animal model, and thus having potential for treating cancer metastasis.

FIG. 10: ZnMoS₄ NPs reduced VEGF expression, without affecting tumor growth of PC3 xenografts. Tumor weights (Panel A) and volumes (Panel B) were measured for eight mice in each treatment, and are shown along with medians for each group. Panel C shows the VEGF gene expression measured in 13 tumor samples. RNA was extracted from frozen mice tumor samples. TaqMan QRT-PCR was performed using VEGF and 18S (normalizer) primers as described in text. Shown are the ratios of 18s normalized VEGF expression in PC3 tumor tissue of mice treated with ZnMoS₄ NPs (high dose and low dose) relative to untreated tumor tissue. Statistical significance was determined by student t-test p<0.05 vs. untreated control.

While in accordance with the Patent Statutes, the best mode and preferred embodiments have been set forth, the scope of the invention is not limited thereto, but rather by the scope of the attached claims.

What is claimed is:

1. Nanoparticles comprising:
a formula M₁MoS₄ or M₁WS₄ where M₁, independently, is, Mg, Ca, Mn, or Fe, and said nanoparticles, independently, have a diameter of from about 4 to about 900 nanometers.

2. The nanoparticles of claim 1, wherein said nanoparticle is a continuous network structure extended by covalent bonds.

3. The nanoparticles of claim 2, wherein said nanoparticle is surface modified with a capping agent comprising a biocompatible polymer, or a water dispersible polymer, or both.

4. A composition for treatment of cancer cells and/or vascular endothelial cells comprising the nanoparticles of claim 3 wherein the particle size of said nanoparticles is from about 10 to about 300 nanometers, and wherein said M₁MoS₄ or M₁WS₄ inhibits angiogenesis by depletion of copper from said cancer cells and/or vascular endothelial cells.

5. A method for producing an angiogenic inhibitor for treatment of cancer and other diseases comprising:
applying nanoparticles of divalent metal M₁, where M₁, independently, is, Mg, Ca, Mn, Fe or Zn, tetrathiomolybdate having the chemical formula M₁MoS₄, or a tetrathiotungstate having the chemical formula M₁WS₄, or both, to an animal.

6. The method according to claim 5, wherein the nanoparticle size of said divalent metal tetrathiomolybdate or said divalent metal tetrathiotungstate, independently, is from about 4 to about 900 nanometers.

7. The method according to claim 6, wherein said nanoparticles are a continuous covalent bonded network,
administering said nanoparticles to a human being having cancer cells and/or vascular endothelial cells, and
inhibiting angiogenesis of said cancer cells and/or vascular endothelial cells.

8. A method for reducing intercellular copper concentrations in a human having cancer cells and/or human vascular endothelial cells, comprising the steps of:
forming a water dispersible covalent network of M₁MoS₄ or M₁WS₄ nanoparticles where M₁, independently, is, Mg, Ca, Mn, Fe or Zn; and
administering an effective amount of said M₁MoS₄ or said M₁WS₄ nanoparticles to said human, said nanoparticles being capable of causing an ion exchange reaction whereby said copper is incorporated in said nanoparticles thereby depleting excessive amounts of copper ions from said cancer cells and/or said vascular endothelial cells, and inhibiting angiogenesis.

9. The method according to claim 8, wherein the particle size of said nanoparticles is from about 4 to about 900 nanometers.

10. The method according to claim 9, wherein the particle size of said nanoparticles is from about 10 to about 300 nanoparticles, and wherein said nanoparticles are not cytotoxic with respect to human vascular endothelial cells.

11. A method for reducing intercellular copper concentrations in a human having cancer cells and/or vascular endothelial cells, comprising the steps of:
forming a water dispersible covalent network of M₁MoS₄ or M₁WS₄ nanoparticles where M₁, independently, is, Mg, Ca, Mn, Fe or Zn, or any combination thereof; and
administering an effective amount of said M₁MoS₄ or M₁WS₄ nanoparticles to effect cellular uptake of said M₁MoS₄ or M₁WS₄ nanoparticles into said cancer cells and/or said vascular endothelial cells and emitting copper therefrom.

12. The method according to claim 11, wherein the particle size of said nanoparticles is from about 4 to about 900 nanometers.

13. The method according to claim 12, wherein the particle size of said nanoparticles is from about 10 to about 300 nanometers.

14. A method for reducing cancer cells and/or vascular endothelial cells in a human, comprising the steps of:
forming a water dispersible extended covalent network of M₁MoS₄ or M₁WS₄ nanoparticles where M₁, independently, is Mg, Ca, Mn, Fe, or Zn, or any combination thereof; and
treating said cancer cells and/or vascular endothelial cells with said M₁MoS₄ or M₁WS₄ compounds and reducing migration of said cancer or human vascular endothelial cells.

15. The method according to claim 14, wherein the particle size of said nanoparticles is from about 4 to about 900 nanometers.

16. The method according to claim 15, wherein the particle size of said nanoparticles is from about 10 to about 300 nanometers, and wherein said nanoparticles are not cytotoxic with respect to human vascular endothelial cells.

17. A method for making M₁M₂S₄ nanoparticles, comprising the steps of:
reacting a basic molybdenum sulfide or a basic tungsten sulfide in a solution of an amide with, independently, a magnesium salt, a calcium salt, a manganese salt, or an iron salt, in an aqueous solution containing a mercapto alkyl acid, and a basic hydroxide and producing said M₁M₂S₄ compound where M₁, independently, is Mg, Ca, Mn, or Fe, and M₂ is molybdenum, or tungsten.

18. The process of claim 17, wherein said basic hydroxide is NaOH, KOH, Ca(OH)₂, or Na₂CO₃, and wherein said salt is a non-zinc salt comprising magnesium acetate, magnesium chloride, magnesium sulfate, magnesium perchlorate, magnesium nitrate; calcium acetate, calcium chloride, calcium sulfate, calcium perchlorate, calcium nitrate; manganese acetate, manganese chloride, manganese sulfate, manganese perchlorate, manganese nitrate; iron(II) acetate, iron(II) chloride, iron(II) sulfate, iron(II) perchlorate, iron(II) nitrate, or any combination thereof.

19. The method of claim 18, wherein said mercapto alkyl acid is 3-mercaptopropionic acid.

20. The method of claim 18, wherein said basic molybdenum sulfide is (NH₄)₂MoS₄ or (NH₄)₂WS₄.
